(19) United States
(12) Patent Application Publication
(10) Pub. No.: US 2006/0089638 A1
(43) Pub. Date: Apr. 27, 2006

(54) Radio-frequency device for passivation of vascular plaque and method of using same

(76) Inventors: Yuval Carmel, Rockville, MD (US); Ron Waksman, Rockville, MD (US); Anatoly Shkvarunets, Rockville, MD (US)

Correspondence Address: SMITH PATENT CONSULTING CONSULTING, LLC, P.O. BOX 2726, ALEXANDRIA, VA, 22301, US

(21) Appl. No.: 11/257,017

(22) Filed: Oct. 25, 2005

Related U.S. Application Data
(60) Provisional application No. 60/622,222, filed on Oct. 27, 2004.

(51) Int. Cl. A61B 18/18

(52) U.S. Cl. 606/41

(57) ABSTRACT

Disclosed herein is a minimally invasive, radio-frequency device and a method for local and regional vascular therapy, more particularly for passivation of atherosclerotic, inflammatory, and/or vulnerable plaque in blood vessels. Radio-frequency devices of the type described herein constitute an important, inexpensive, disposable, minimally invasive approach for passivation or removal of plaques in various parts of the human body, and, as such, have cardiological applications, such as the treatment of coronary atherosclerosis, as well as other applications, such as the treatment occluded blood vessels in the legs and extremities.

### PRIORITY

This application claims the benefit of U.S. Provisional Application Ser. No. 60/622,222 filed Oct. 27, 2005, the contents of which are incorporated by reference herein.

### TECHNICAL FIELD OF THE INVENTION

The present invention relates to local and regional vascular therapies, more particularly, to a minimally invasive, radio-frequency device and method for passivation of atherosclerotic, inflammatory and vulnerable plaque in blood vessels. Radio-frequency devices of the type described herein constitute an important, inexpensive, disposable, minimally invasive approach for passivation or removal of plaques in various parts of the human body, and, as such have cardiological applications, such as the treatment of coronary atherosclerosis, as well as other applications; such as the treatment occluded blood vessels in the legs and extremities.

### BACKGROUND OF THE INVENTION

Coronary atherosclerosis constitutes the fifth leading cause of global disease burden and the leading cause in developed societies (1). While a decrease in the mortality rate from coronary disease has arisen in the past few decades, due to therapeutic advances and changes in lifestyle in the general population, it is expected that, in spite of these advances, over the next two decades ischemic heart disease will become the leading cause of morbidity and mortality in the world, surging past infectious diseases (1). Coronary atherosclerotic disease is predominantly an asymptomatic process. Growth of coronary plaque presents clinically as angina when coronary flow is decreased. This clinical syndrome can be stable for many years; and a myriad of medical, interventional, and surgical options are available for its treatment. On the other hand, sudden rupture of a plaque triggers the development of an acute coronary syndrome, such as unstable angina, myocardial infarction or sudden death. Reduction in acute coronary events requires interventions that affect the mechanisms leading to formation of atherosclerotic lesions, as well as the molecular events that precipitate acute myocardial infarction.

Data from clinical trials indicate that it is the vulnerability of atherosclerotic plaque to rupture, rather than the degree of atherosclerosis that is the primary determinant of most acute coronary syndromes, including sudden cardiac death, acute myocardial infarction, and unstable angina (3-6). The morphologic features of thin cap atheromas that predict rupture are unknown, but from studies of ruptured plaques, scientists know the characteristics of a plaque that is vulnerable to rupture include such a thin fibrous cap that separates the circulation from procoagulants in the plaque's lipid core as well as an increase in the number of inflammatory cells, such as macrophages, and a relative paucity of vascular smooth muscle cells (7-12). There is some evidence that a speckled pattern of calcification is also associated with vulnerability to rupture (13-16). Targeted therapy for the purpose of stabilizing coronary lesions that are prone to rupture is an attractive way to prevent the complications associated with plaque rupture.

An electromagnetic field fluctuating at radio frequencies can be used to produce regional hyper-thermia within a body (17). The area of heating can be focused by manipulation of the position and shape of the active electrode (18). The introduction of an invasive probe into the area of interest allows current to flow into the tissue. Ions in the tissue move according to the electric field distribution, creating current density, resulting in ohmic heating in the tissue, and a temperature rise in the proximity of the probe. The net amount of heat dissipated (absorbed) in the region being treated (i.e., the region proximate to the probe tip) is the difference between the heat produced by radio-frequency current flow and the heat lost by conduction and convection. As a result, the temperature rise and distribution in the treated region is a complex function of RF power distribution, thermal properties and electrical conductivity of tissue, blood and time.

Radio-frequency already has multiple therapeutic uses in medicine. Some of the uses most relevant to plaque passivation are include: ablation of arrhythmogenic cardiac tissue (19, 20) recanalization of vascular occlusions (21), and coagulation of blood vessels (22).

### BRIEF SUMMARY OF THE INVENTION

From studies of ruptured plaques, it is known that the characteristics of a plaque that is vulnerable to rupture include a thin fibrous cap separating the circulation from procoagulants in the plaque's lipid core, an increased number of inflammatory cells, such as macrophages, and a relative paucity of vascular smooth muscle cells (7-12). The present invention relates to the discovery that application of radio-frequency energy to an atherosclerotic, inflammatory and/or vulnerable plaque transforms the structure of the plaque into relatively rigid fibrotic lesions that are less prone to rupture and embolization. As such, acute conditions such as myocardial infarction may be prevented. Accordingly, the subject of this invention is a minimally invasive, radio-frequency device and a method for passivation of plaque in blood vessels having utility in treating cardiological conditions, coronary atherosclerosis, occluded blood vessels in the legs, as well as other applications, both human and veterinary. More specifically, the subject of this invention is a catheter-based radio-frequency device for use in local and regional vascular therapy, more particularly for passivation of atherosclerotic, inflammatory and/or vulnerable plaque in blood vessels. Such devices by definition operate at low, radio-frequency power levels and are therefore safe, in the sense that they are less likely to cause side effects and damage to the surrounding blood vessel walls or tissue of the patient.

Radio-frequency based devices of the type described in this invention constitute an important, inexpensive, disposable, minimally invasive approach for passivation of plaques in various parts of the human body. The radio-frequency active element (distal tip, probe) is an exposed conductor mounted at the distal end of an otherwise electrically insulated cardiac catheter which act as a delivery system. The delivery system as well as the radio-frequency active element are either radiopaque or contain marker bands at the appropriate locations. Both the catheter and the active element are small enough such that they can be introduced into the blood vessels (vascular tree) without blocking the natural blood flow. The location of the active element may be continuously monitored by a suitable imaging system, such as an x-ray imaging system or fluoroscope. When the active tip reaches the desired location to be treated in the blood vessel, the apparatus is energized by activating a radio-frequency control unit (generator), typically via a foot switch connected to the control unit or an activation button in the handset of the device.

The heat produced by the radio-frequency device of the present invention is localized and focused in close vicinity to the one ore more active elements mounted to the distal end of the catheter. The degree of heat focusing depends on the geometry of the active element and on the electrical properties of the blood, tissue and plaque in its vicinity, as well as the degree of heat loss from the area (due to blood flow and conduction of heat). By properly positioning the active element in the blood vessel, and by properly choosing the radio-frequency exposure time and power, the apparatus can have little or no deleterious effects on the surrounding patient tissue, and the plaque can be passivated by selectively reducing the lipid core and/or macrophages. It is envisioned that moving, or repositioning the catheter (manually or automatically, in steps or continuously) during the procedure will enhance the passivation. In addition, by properly choosing other exposure time and power, the plaque can be evaporated and removed.

Devices based on the principles of this invention can be both monopolar or bipolar.

In order for a vascular plaque to be passivated, it should be heated (possibly to ˜50° C.), without overheating of the blood vessel walls. In other words, selective heating is desirable and beneficial, so that a desired temperature distribution is established in the region to be treated. Selective heating of the plaque can be achieved by taking advantage of the different electrical and thermal properties of the plaque, tissue, blood and catheter. Accordingly, the present invention relates to a radio-frequency device, having an active element (i.e., an exposed tip made of an electrically conductive material, such as metal) responsible for generating the desired, local distribution of the RF energy in the region where the procedure is to be performed. In operation, the active element is placed in close proximity, inside the blood vessel, to the region where the lumen of the blood vessel is constricted by the presence of plaque, and then energized. The RF current flows through the surrounding media (blood, plaque, and tissue) and locally deposits RF electrical energy. This leads to local heating in the media around it. The temperature distribution generated in the media depends on the RF power distribution, time of exposure and the thermal and electrical properties of the material involved. As a result, the highest rate of temperature rise is near the active element (probe tip).

After the catheter, and particularly the radio-frequency active element at the tip, is positioned in the desired location within the blood vessel, there are two possible modes of operation for plaque passivation:
1) The active area (i.e., one or more active elements) of the device is in intimate contact with the plaque, and
2) The active area of the device is not in intimate contact with the plaque.
   In the first case, the temperature of the plaque rises faster than the temperature of the blood vessel wall, due to differences in their conductive properties. Properly chosen RF power and activation times will allow selective passivation of the plaque. For example, in this context, the power level applied may be less than 50 W, preferably less than 25 W, more preferably less than 10 W, even more preferably less than 5 W. The radio-frequency energy applied may range from 100 kHz to 10 MHz, though it preferably ranges from 300 kHz to 4-6 MHz. Regarding the duration of RF energy application, the process typically will range up to 20 seconds per treatment site.

In the second case, the tip is surrounded by streaming blood. The induced RF current flows through the moving blood, plaque and blood vessel tissue. The blood streaming through the region near the probe tip substantially reduces (cools) the temperature in this region. The RF energy is accumulated in the stationary plaque region, again heating this region more than any other region. The temperature of the blood vessel wall in this case is again substantially lower than the plaque, thereby again creating selective heating of the plaque.

In addition, there is a third mode of operation, namely a combined thermal passivation and plaque removal (ablation). This mode of operation takes place when the plaque region is in contact with the active area of the device, and somewhat higher RF power levels are chosen. In this mode, “overheating” of the media around the probe's tip will occur. As a result, some fraction of the liquid is locally evaporated, and steam bubbles “insulate” the tip of the probe. Accordingly, the voltage at the tip increases, creating large enough electric fields in the bubbles, which, in turn lead to electrical breakdown and the formation of ionized channels in the bubbles. These ionized channels additionally focus the RF power and evaporate and disintegrate any biological material in its immediate vicinity. At this increased power level, two mechanisms for treatment of plaque are active simultaneously; heating, which is the “thermal” passivation described earlier, and also partial ablation (evaporation). When ionized channels in the bubbles are formed, both the thermal and ablation mechanisms are active simultaneously. The transition between thermal passivation and the combined thermal/ablation mechanisms depends not only on the RF power level, but also on the characteristics and operating regime of the RF generator. Note that the third mode of operation is particularly useful for treating blood vessels with medium or large degree of lumen blockage by plaque.

As noted above, the method of the present invention may further involve the step of moving, or repositioning the device during the procedure so as to enhance passivation and increase the area being passivated. This movement may be achieved through manual manipulation or, alternatively, may be automated. The movement may be stepwise (i.e., interrupted) or continuous, and may comprise linear (translational) or rotational movement or a combination of the two. In terms of linear movement, the device may be pulled or pushed, continuously or in a stepwise fashion, in a single direction to cover a range of plaque during period of energy activation, or, alternatively, one may oscillate the device back and forth over a central plaque position.

Accordingly, it is an object of the present invention to provide a method of local or regional vascular therapy for atherosclerotic, inflammatory, and/or vulnerable plaque in a blood vessel in a subject in need thereof comprising the steps of:
(a) introducing a radio-frequency device having one or more active electrodes at its distal end into a blood vessel;
(b) positioning one of the active electrodes in close proximity to the plaque;
(c) applying low power radio-frequency energy to the active electrode for a controlled amount of time so as to passivate the plaque by selectively heating the plaque while minimizing the heat generated in the blood vessel wall and surrounding tissue, wherein said selective heating results in transformation of the plaque into mechanically stable fibrotic lesions having a reduced risk of thrombosis.

The method may further include the steps of (d) repositioning the device during step (c) so as to enhance passivation. Such repositioning may be achieved through manual manipulation or, alternatively, may be automated.

In a preferred embodiment, the radio-frequency device utilized comprises: at least one active electrode that protrudes beyond the distal end of an elongated electrical insulator sleeve, wherein at least one conductive wire, connected to one of the active electrodes at one end and an external radio-frequency control unit at its other end, is disposed within the insulator sleeve. The active electrode may be monopolar or bipolar.

In another preferred embodiment, the radio-frequency device for passivation of vascular plaque comprises: a first active electrode connected to a conductive wire, an insulator sleeve coaxially disposed about the conductive wire, and a second active electrode coaxially disposed and mounted to the distal end of the insulator sleeve yet disconnected from the external source of radio-frequency energy, wherein the distal end of the first electrode extends beyond the distal ends of the insulator sleeve and second electrode.

In another preferred embodiment, the radio-frequency device for passivation of vascular plaque comprises: a first active electrode connected to a conductive wire and an insulator sleeve disposed about the electrode, wherein the distal end of the insulator sleeve is slidably disposed about the active electrode and is provided with a series of lateral openings that control of the direction of energy applied from the active electrode to the plaque.

In yet another preferred embodiment, the radio-frequency device for passivation of vascular plaque comprises: an active electrode connected to a conductive wire and an insulator sleeve disposed about the conductive wire, wherein the insulator sleeve is provided with one or more lateral openings and the active electrode includes one or more projections, each of which extend through one of the lateral openings.

These and other objects and features of the invention will become more fully apparent when the following detailed description is read in conjunction with the accompanying figures and examples.

### BRIEF DESCRIPTION OF THE DRAWINGS

FIG. 1 is a schematic diagram showing the radio-frequency control unit, the radio-frequency probe, and the hand and foot switches for activation. Also shown is a return electrode, referred to herein as a dispersive pad. The device based on the present invention can be either monopolar, as shown in this figure, or bipolar.

FIG. 2 (a) is a schematic diagram showing the delivery system element inserted into the patient's blood vessel, while its position is monitored by an imaging system, such as a fluoroscope (not shown). FIG. 2 (b) is a schematic diagram showing the space around the exposed tip (active area) of the radio-frequency device of the present invention, for passivation of vascular plaque, inside a blood vessel wherein the lumen is partially blocked by plaque.

FIG. 3 is a schematic diagram of an illustrative radio-frequency device for passivation of vascular plaque device, including the active element (tip, conductor), the insulator, the blood, the plaque and the blood vessel wall.

FIG. 4 depicts exemplary numerical calculations showing the contour lines of constant electric field/current density in the vicinity of the active element and in the plaque during activation.

FIG. 5 depicts exemplary numerical calculations demonstrating the principle of selective heating. The temperature distribution in a cross section inside and outside a blood vessel partially blocked with plaque in the vicinity of the active electrode tip, after 0.5 sec of RF activation is shown. For the purposes of this example, blood flow rate is estimated to be 1 cm/second.

FIG. 6, parts (a) to (h), shows various geometries of the active element (tip) of the radio-frequency device envisaged in the context of the present invention, based on a monopolar, azimuthally symmetric design. Note that the devices based on the principles of this invention can also be equipped with a front guide coil, as shown in parts (f) and (g).

FIG. 7, parts, (a) to (c), shows various geometries of the active element envisaged by the present invention, based on a monopolar, non-symmetric design. Note that the devices of the present invention can also be equipped with a stainless steel or platinum guide coil, as shown in (f) and (g) of FIG. 6.

FIG. 8, parts (a) to (d), shows various coaxial geometries of the active element, based on a bipolar, azimuthally symmetric design, contemplated by the present invention. Note that the devices of the present invention can also be of non-azimuthally symmetric designs, and equipped with a front guide coil as shown in FIG. 6 (f) and (g).

FIG. 9, parts (a) to (d), shows further contemplated coaxial geometries of the active element of the present invention, based on a bipolar, azimuthally symmetric design. Note that the devices based of the present invention can also be of non-azimuthally symmetric designs, and equipped with a front guide coil as shown schematically in FIG. 6 (f) and (g).

FIG. 10, parts (a) to (c), shows still further contemplated geometries of the active element of the present invention, based on a bipolar or multi-polar, multiple electrode design. Note that the devices based on the principles of this invention can also be of non-azimuthally symmetric designs, and equipped with a stainless steel or platinum guide coil, as shown schematically in FIG. 10(c).

FIG. 11 (a) shows a schematic sketch of the active element of the device of the present invention, equipped with an aspiration and/or irrigation port. Both bipolar and monopolar devices aspiration/irrigation versions are contemplated herein. FIG. 11 (b) shows a schematic sketch of the active element equipped with a temperature-sensing element. Again, both monopolar and bipolar devices are contemplated herein.

FIG. 12 shows further geometries of the active element. The active area in (a) is a monopolar device having multiple exposed protrusions or projections. FIG. 12 (b) is an example of a monopolar electrode inserted in a movable insulator, allowing the exposure of different segments of the active electrode and the medical treatment of different regions of the blood vessel according to the specific needs of the patient. FIG. 12 (c) is an example of a device based on multiple, electrically independent electrodes. The electrodes can be activated independently, sequentially of simultaneously. FIG. 12 (d) is an example of a device equipped with one or more detachable (not connected) electrodes. Note that the devices can also be of non-azimuthally symmetric designs, and equipped with a front guide coil. Other variations are also envisaged.

FIG. 13, parts (a)-(b), shows schematic diagrams illustrating various methods for delivering and guiding the radio-frequency device of the present invention inside blood vessels. In (a), the device is inserted inside a flexible tube to be inserted into the blood vessel. In (b), the device is equipped with a flexible guide coil. In (c), the device glides along a guide wire with the aid of a front mounted guiding fixture. In (d), the device glides along a guide wire with the aid of a side mounted guiding fixture.

### DETAILED DESCRIPTION OF THE PREFERRED EMBODIMENTS

Unless otherwise defined, all technical and scientific terms used herein have the same meaning as commonly understood by one of ordinary skill in the art to which this invention belongs. In case of conflict, the present specification, including definitions, will control.

The words “a”, “an”, and “the” as used herein mean “at least one” unless otherwise specifically indicated.

The present invention makes reference to “atherosclerotic”, “inflammatory”, and “vulnerable” plaque. Atherosclerosis is commonly referred to as a “hardening” or “furring” of blood vessels, but this is an oversimplification. Vascular lesions, known as atheromas, develop in the vessel wall and, in late stages, may suddenly rupture (e.g., a vulnerable plaque or acute inflammatory plaque) and reduce or totally stop blood flow in the lumen (i.e., stenosis), leading to damage of the tissue downstream which has lost needed blood flow (i.e., ischemia).

In the context of the present invention, a vulnerable plaque is an unstable inflammatory plaque which is particularly prone to rupture and then to either embolize or to occlude the artery it occupies, thereby producing sudden acute events, such as heart attack or stroke. Importantly, it is vascular biology and not the degree of stenosis that determines plaque stability. In general, the process of plaque destabilization begins with endothelial dysfunction against a background of inflammation. The vulnerable plaque typically has three hallmark histologic features: (i) a large, highly thrombogenic, lipid core occupying more than 40% of the plaque volume; (ii) an abundance of inflammatory cells; and (iii) a thin fibrous cap that lacks proper collagen and smooth muscle cell support.

The acute clinical event is precipitated by the formation of an intimal, platelet-rich thrombus followed in some cases by a fibrin-red cell intraluminal thrombus. Established risk factors of plaque vulnerability include:
increased lipid content (>40%)
reduced collagen content in a thinned fibrous cap
increased inflammatory cell infiltration, commonly macrophages
increased expression of matrix degrading metalloproteinases (MMP)
reduced expression of tissue inhibitor of MMP (TIMP)
increased concentrations of macrophage colony stimulating factor (M-CSF)
haemodynamic shear stress.
   Treatment of these risk factors may reduce the probability of plaque erosion or rupture and subsequent thrombus formation and acute coronary syndrome (ACS).

The present invention is primarily directed to plaque stabilization and passivation, as distinguished from plaque ablation and removal. In the context of the present invention, plaque stabilization is defined by any intervention or interaction which, by causing a change in either the structure, content or function of an atherosclerotic plaque and/or the overlying endothelium, will either prevent or reduce the severity of erosion or rupture. Plaque passivation is defined as any intervention that decreases the thrombogenicity of the endoluminal oriented vascular surface. In the context of the present invention, selective heating of the vascular plaque, through local application of low power radio-frequency energy, results in a modification of the plaque structure and/or content (e.g., a transformation of the inflamed tissue into fibrotic lesions and/or a selective reduction of the lipid core and/or macrophages, each of which translate into an increase in plaque density, mechanical stability and/or rigidity). The upshot of passivation is a reduction in the risk of subsequent rupture and thrombosis, which, in turn, correspond to a reduction in the risk of acute events such as myocardial infarction.

The radio-frequency devices of the present invention have both medical and veterinary applications. Accordingly, the term “subject” as used herein refers to both humans and animals, more preferably mammals.

Hereinafter, reference is made to the accompanying drawings which depict, by way of illustration, specific embodiments of the invention and its practice. These embodiments are described in sufficient detail to enable those skilled in the art to practice the invention, and is to be understood that other embodiments may be utilized, and that structural, logical and electrical changes may be made without departing from the spirit and the scope of the present invention. In the drawings, like elements are designated by like reference numerals when appropriate.

The words exposed electrode, probe tip, distal tip, and active electrode are used interchangeably to describe the exposed, non-insulated, electrically conductive area of the device which is in contact with the blood, plaque and/or tissue. Activating, or energizing, the active tip, from an external radio-frequency control unit, will lead to current flow in the vicinity of the active tip and surrounding region, and passivation of vascular plaque, which together generate a clinically beneficial effect for the patient. The heat produced by radio-frequency device of the present invention is localized and focused in close vicinity to the active element(s), mounted at the distal end of the catheter. The degree of heat focusing depends on the geometry of the active element(s) and on the electrical properties of the blood, tissue and plaque in its vicinity, as well as the degree of heat losses from the area (due to blood flow and conduction of heat). By properly positioning the active element in the blood vessel, and by properly choosing the radio-frequency exposure time and power, the apparatus will have no deleterious effects on the surrounding patient tissue, and the plaque can be selectively heated and passivated by selectively reducing the lipid core and/or macrophages, converting the plaque into mechanically stable fibrotic lesions that are less prone to rupture. It is envisioned that moving, or repositioning, the catheter during the procedure could enhance the passivation and will also allow the medical personnel to effectively and quickly treat large regions. In addition, by properly choosing other exposure time and power settings, the plaque can be evaporated and/or removed.

As noted above, devices based on the principles of this invention can be both monopolar or bipolar. Referring now to the drawings:

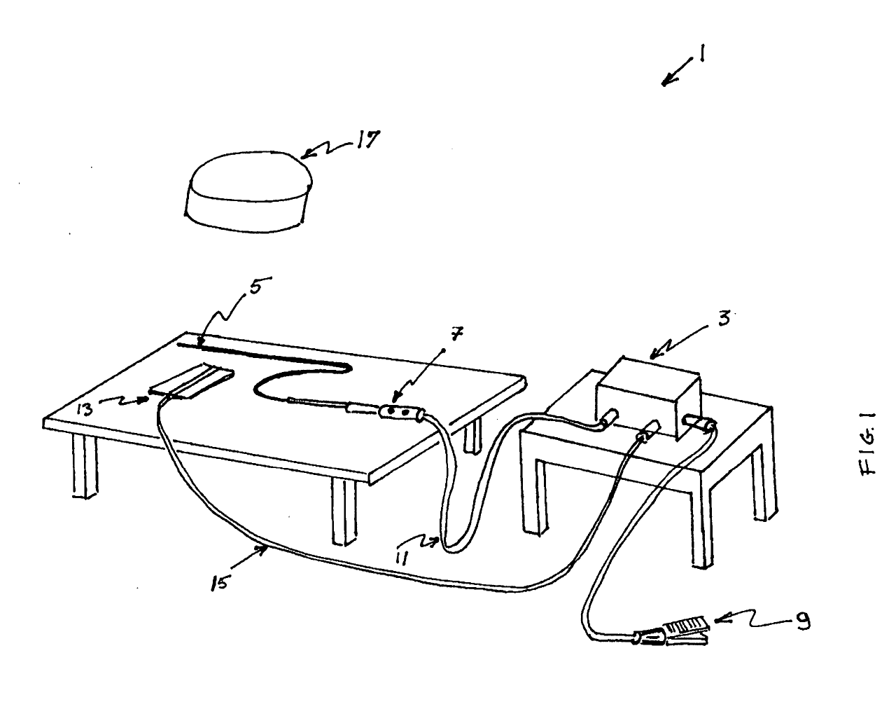

FIG. 1 shows a schematic diagram of a catheter based radio-frequency system 1 for use in the context of local and regional vascular therapy, more particularly for passivation of vascular plaque. The system is composed of a radio-frequency control unit 3, a radio-frequency catheter or probe 5, a hand switch 7, a foot switch for activation 9. Also shown is a catheter electrical cable 11, a return electrode 13 (also known as a dispersive pad), an electrical cable 15 connecting the return electrode 13 to the radio-frequency control unit 3 and an imaging system 17 to monitor the position of the catheter in the patient body (not shown). The radio-frequency device of the present invention can be either monopolar, as shown in this figure, or bipolar. In the case of bipolar devices, no return pad is needed.

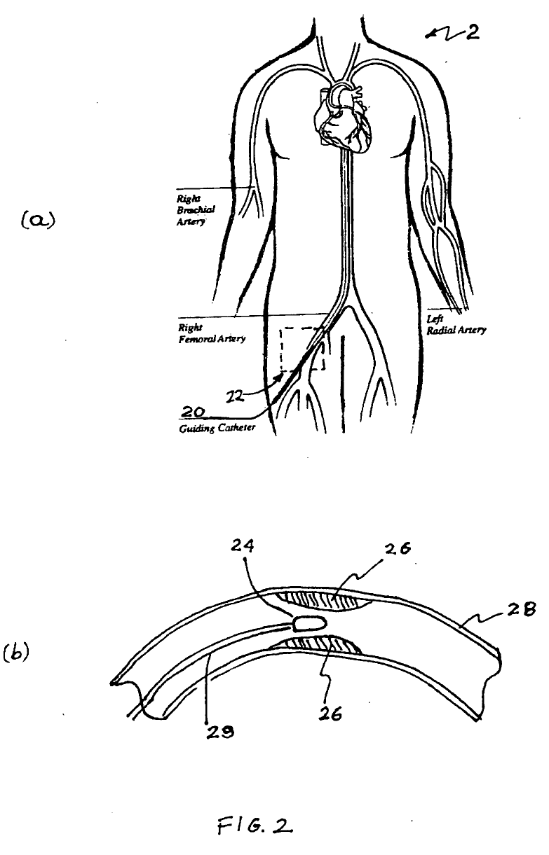

FIG. 2 (a) shows schematically a human body 2 with some of the blood vessels labeled. One possible way to deliver the device of the present invention to the desired position within the vasculature is through a small opening in a main blood vessel. The schematic diagram of FIG. 2(a) shows the delivery system 20 (labeled as guiding catheter) being inserted into the patient's blood vessel, while its position is carefully monitored by a well known imaging system, such as fluoroscopy (not shown), available from various vendors like Phillips and others. An expanded view of the region marked by a dashed line 22 in FIG. 2(a) is shown in FIG. 2(b). It shows schematically the area around the tip (i.e., the active area) 24 of an embodiment of the radio-frequency device for passivation of vascular plaque and wire 29 connecting the active area 24 to the external radio-frequency control unit (3 in FIG. 1). The active area 24 of the radio-frequency system 1 for passivation of vascular plaque is delivered by the medical personnel to the vicinity of the area inside the blood vessel 28 where the plaque 26 is located, with the help of an imaging system (not shown).

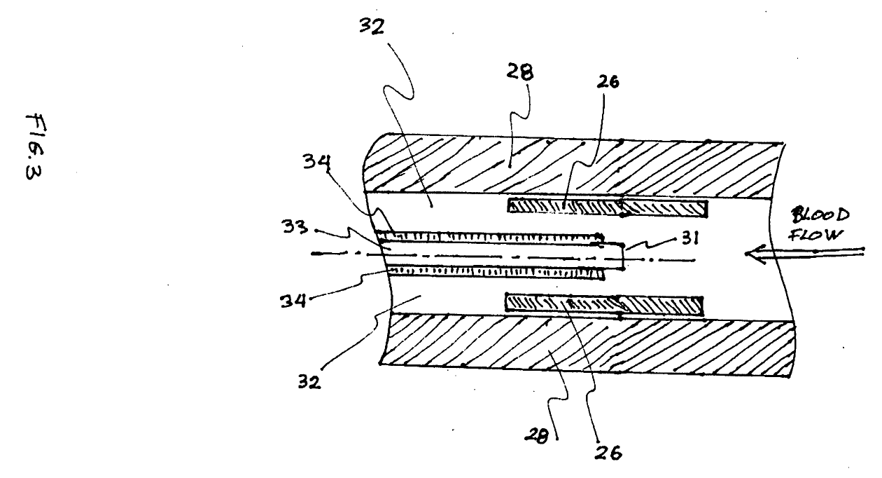

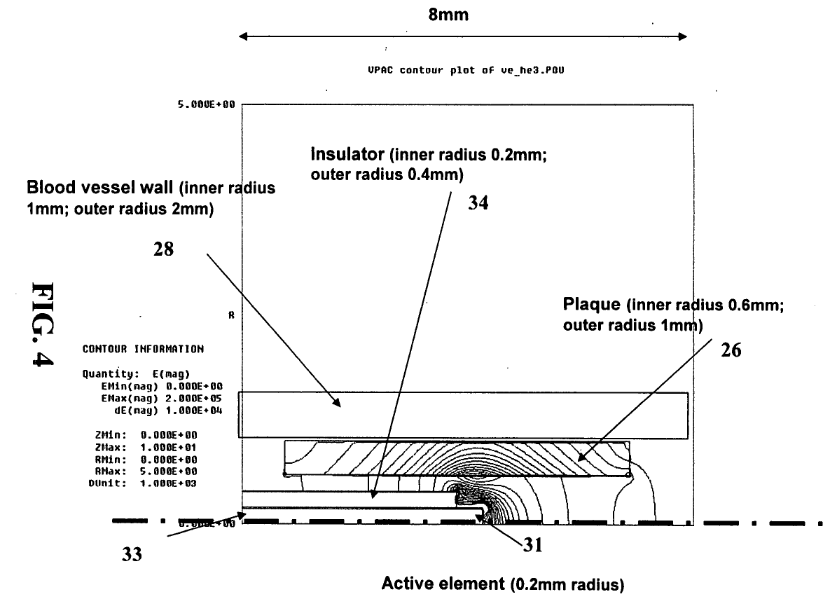

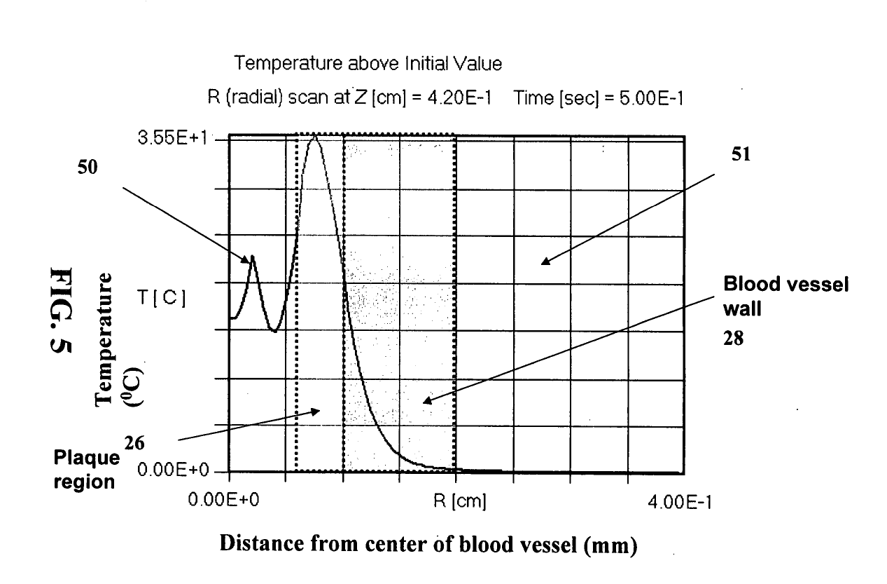

FIG. 3 shows a schematic diagram of the active area (tip) of an embodiment of the radio-frequency device of the present invention, for use in the context of local or regional vascular therapy, more particularly for passivation of vascular plaque. The exposed active area 31 of the device is navigated through the blood vessel 28 while being monitored by an imaging system 17 and brought to the vicinity of the plaque area 26 inside a blood vessel 28. The active area 31 is connected to the radio-frequency control unit 3 via an electrical conductor 33 which is coated with an insulator 34 made of a flexible dielectric. The exposed active area 31 as well as the electrical insulation 34 of the electrical connector 33 are all immersed in the blood 32 flowing in the blood vessel 28. The schematic diagram in FIG. 3 is shown for illustration purpose only. It will be used as an example for the purpose of numerical modeling, the results of which are shown in FIGS. 4 and 5, in order to demonstrate the principle of selective heating of the plaque.

An example of a numerical three-dimensional, azimuthally symmetric calculation showing the lines of constant electric field/current density in the vicinity of the exposed tip 31 of the device is shown in FIG. 4. The figure is symmetric around the centerline, and shows only half of the plane around the centerline. The calculation shows concentration of radio-frequency energy in the vicinity of the active element 31 and in the plaque region 26 during activation. The dimensions provided in FIG. 4 are given in millimeters and are for illustrative purposes only.

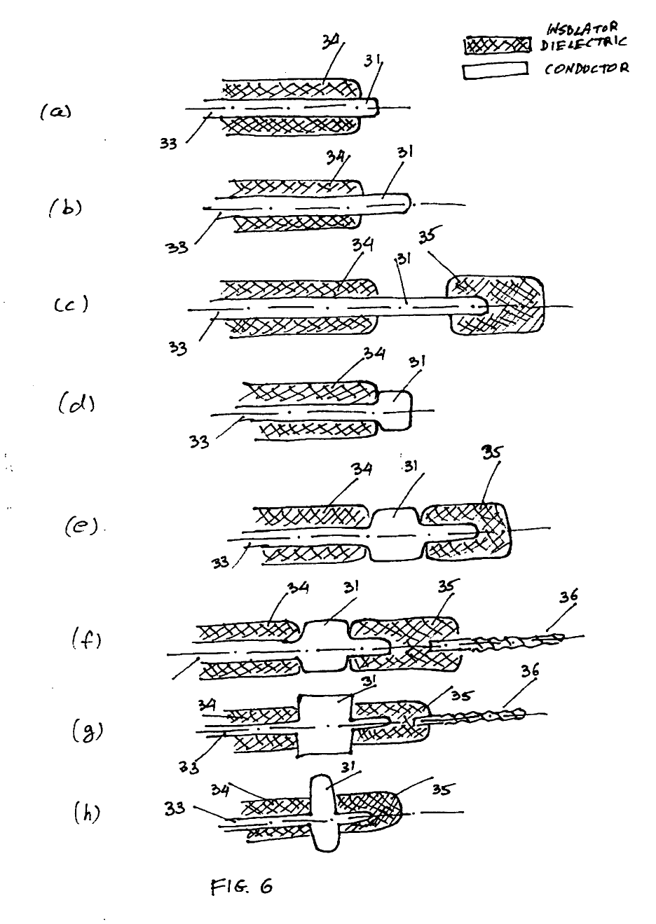

FIG. 5 further demonstrates, through a numerical example, the principle of selective heating. The figure shows the temperature rise 50 as a function of the distance away from the center of the blood vessel. The temperature distribution is shown in a cross section inside and outside a blood vessel partially blocked with plaque after 0.5 sec of RF activation, with a blood flow rate of 1 cm per second. Because of symmetry, only half of the plane around the centerline is shown. From the figure, one can see that the highest temperature rise is achieved in the plaque region, which then leads to plaque passivation. This process is referred to as selective heating, wherein the radio-frequency energy is concentrated mostly in the plaque region, as desired. The degree of heating in the blood vessel wall 28 and in the tissue around it 51 is minimal. FIG. 6 show eight exemplary embodiments of the front active area (tip) of the radio-frequency device of the present invention, based on a monopolar, azimuthally symmetric design. Note that activating, or energizing, the device tip will lead to heating and passivation of vascular plaque, and thereby generate the clinically beneficial effect for the patient. FIGS. 6 (a) and (b) show an active, exposed electrode 31, an electrical insulator 34 and an electrical conductor (e.g., a wire or metal catheter) 33 connecting the electrode to the radio-frequency control unit. The electrode 31 can slightly protrude beyond the front end of the insulator 34, as shown in FIG. 6 (a), or, alternatively, may be flush with the insulator. It can also extend well beyond the front surface of the insulator 34, in order to be able to treat long sections inflicted with plaque. FIG. 6 (c) shows yet another embodiment where the active electrode 31 is connected at its distal end to a second insulator 35. Yet another embodiment is shown in FIG. 6 (d), wherein the active electrode 31 has a diameter larger than that of the conducting wire 33. The embodiment shown in FIG. 6 (e) is similar to that of FIG. 6 (c), wherein the active electrode 31 extends to a second insulator 35; however, in the depicted embodiment, the active tip 31 has an outside diameter slightly less than that of the insulators 34 and 35. FIGS. 6 (f), (g) and (h) are variations of the designs shown in (a) through (e), with the exception that the exposed active electrode can extend radially beyond the outside diameter of the insulators 34 and 35. Note that the any embodiment of a devices of the present invention may be equipped with a front guide coil 36, as shown in (f) and (g), that can be used to help navigate the device inside the patients blood vessels, under the guidance of the imaging system. Accordingly, the front guide coil may be made of a material that will be clearly visible under the imaging system. For example, the front guide coil can be made of stainless steel, platinum, or the like so as to allow it to be visualized using an imaging system such as a fluoroscope.

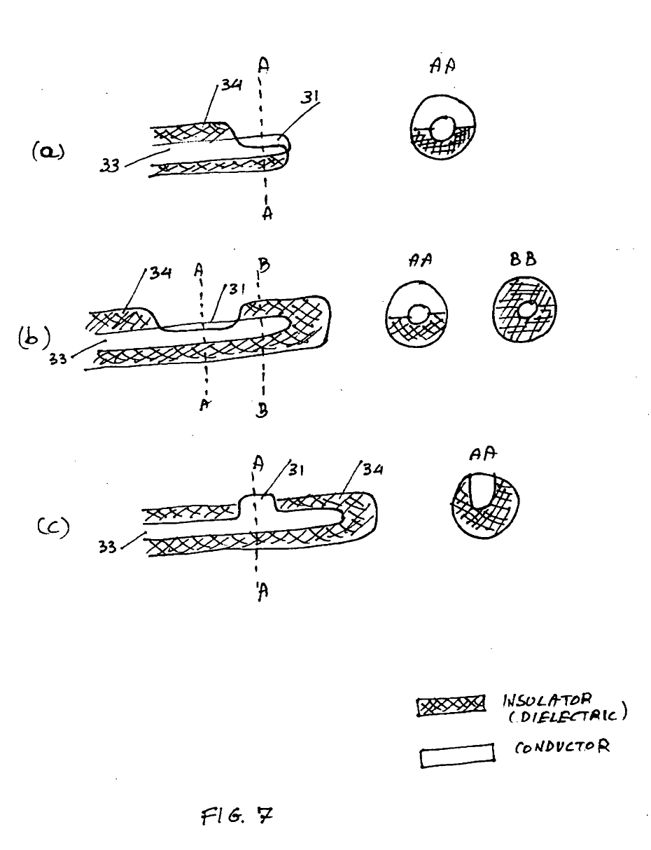

FIGS. 7 (a), (b) and (c) depict additional illustrative embodiments of the active element of the present invention, based on a monopolar, non-symmetric design. More particularly, the embodiments of FIGS. 7 (a)-(c) depict an electrical insulator 34 having a non-symmetrical distal end. For example, in FIG. 7(a), a portion of the distal tip of the electrical insulator 34 is beveled, chamfered, or cut away so as to control the direction of RF energy applied, for example, to one side of the vessel or the other. Similarly, FIGS. 7(b) and (c) depict an electrical insulator 34 having at least one lateral opening through which RF energy may focused. The active electrode may be contained within the insulator or, alternatively, may be provided with a lateral projection that protrudes through the opening. As with the embodiments of FIG. 6, in the context of the embodiments of FIG. 7, the electrode 31 can slightly protrude beyond the insulator, or, alternatively, may be flush with the insulator. It can also extend well beyond the front surface of the insulator in order to be able to treat long sections inflicted with plaque. These devices will be especially useful for treating regions in blood vessels where plaque has accumulated on only one side of the vessel (i.e., an asymmetric plaque). The active electrode 31 and the electrical insulator 34 may be non-cylindrically symmetric, allowing for plaque passivation in preferred regions. The active electrode is energized by radio-frequency energy supplied by a radio-frequency control unit (not shown), via the electrical connection 33. Note that all the devices based on the principles of this invention can also be equipped with a front guide coil as shown in FIG. 6 (f) and (g).

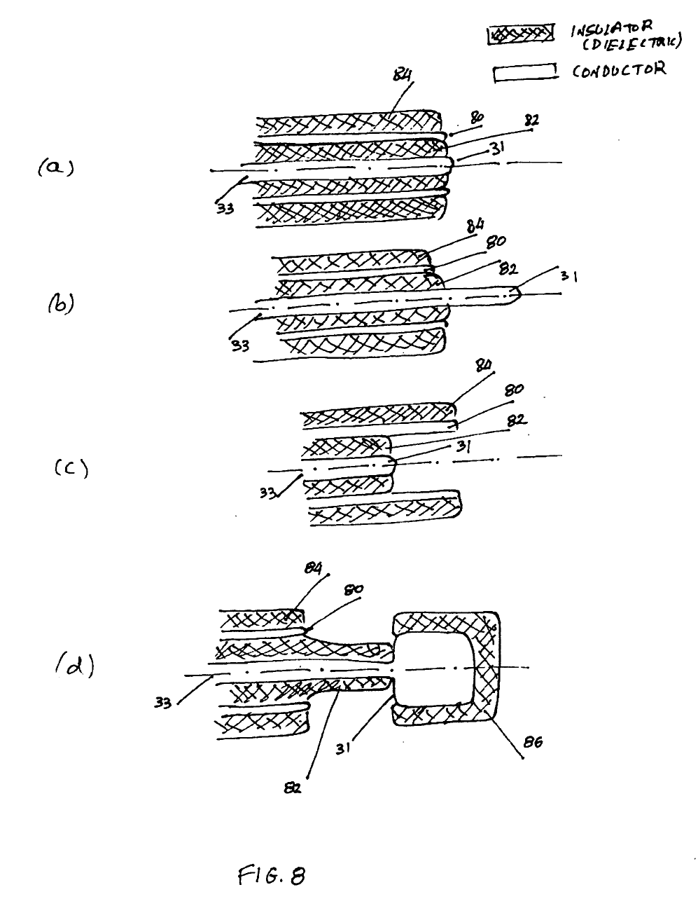

FIGS. 8 (a) to (d) show four exemplary coaxial embodiments of the distal active element of the present invention, based on a bipolar, azimuthaly symmetric design. For bipolar devices, according to the principles of the present invention, the device is equipped with at least one active electrode 31, at least one passive return electrode 80 electrically insulated from each other using a first insulator 82 and a second insulator 84. When the device is activated, radio-frequency current flows between the active electrode and the return electrode. An additional embodiment is shown in FIG. 8 (d), wherein the active electrode 31 includes another electrical insulator 86. Note, the roles played by the active electrode 31 and the return electrode 80 may be easily reversed. Connection to the external radio-frequency unit (i.e., element 3 shown in FIG. 1) is made via the electrical connection 33 connected to the active element 31, and an additional electrical connection (not shown) connected to the return electrode 80. No dispersive pad is needed for the bipolar devices.

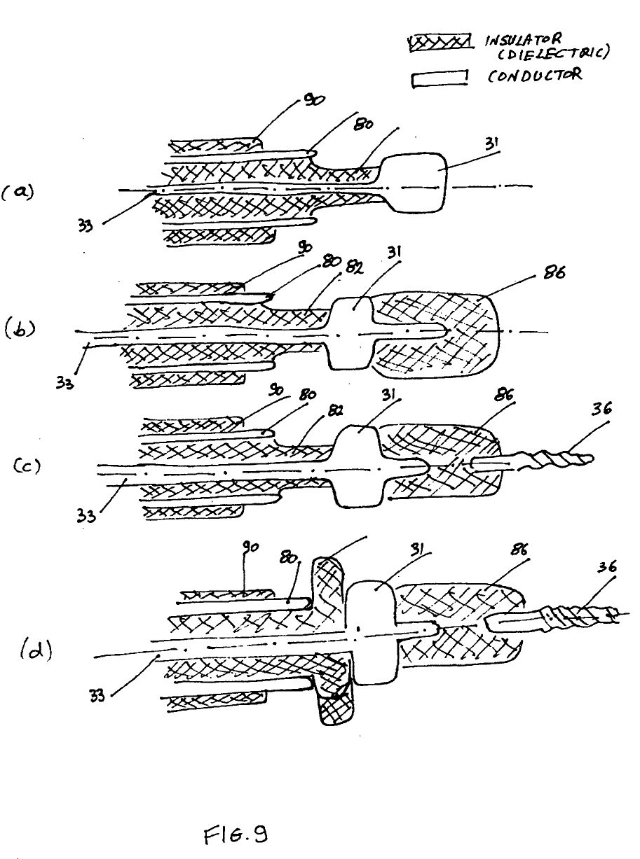

Additional bipolar, coaxial embodiments are shown in FIGS. 9 (a) to (d), all based on azimuthally symmetric designs. Note that the devices based on the principles of the present invention can also be of non-azimuthally symmetric designs, and equipped with a front guide coil of the type shown schematically in FIG. 6 (f) (g). The embodiments of FIG. 9 include three or four different zones of insulation. For example, 82 is the insulation around the active electrode 31, and between the active electrode 31 and the return electrode 80. A third zone of insulation 90 partially covers the return electrode 80. Yet another zone of insulator 86 is attached to active electrode 31. Note that non-azimuthally symmetric embodiments are also contemplated, and that the devices based on the principles of this invention can also be equipped with a front guide coil 36 as shown in FIGS. 6 (f) and (g).

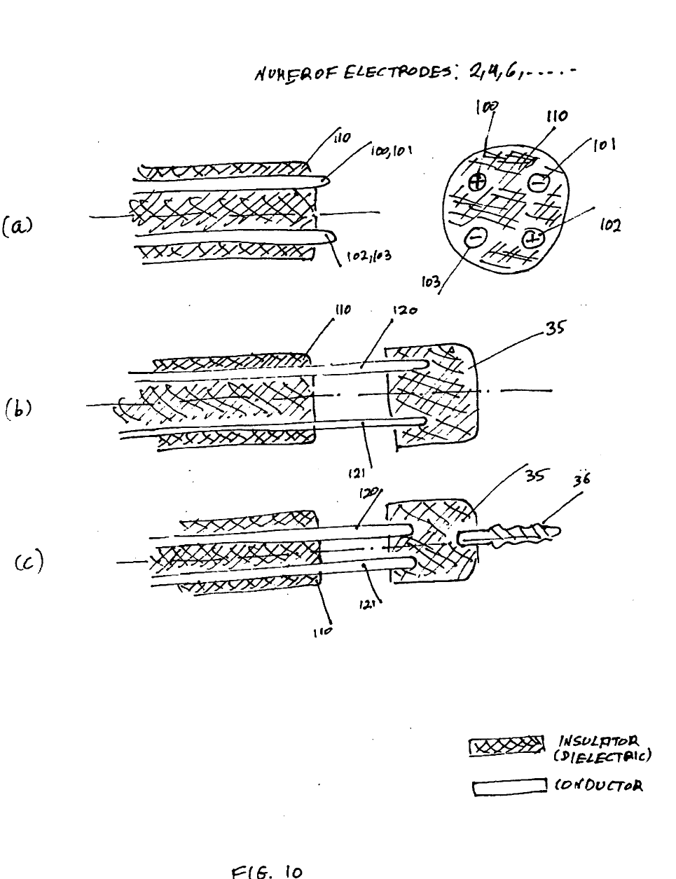

FIGS. 10 (a) to (c) show still further embodiments of the present invention, based on bipolar or multi-polar electrode designs. In the embodiment depicted in FIG. 10(a), the number of electrodes is N=4; however, it can be any even number, i.e., N=2, 4, 6, 8, . . . etc. FIG. 10 (a) shows an embodiment incorporating four electrodes 100, 101, 102, 103, insulated by insulator 110. FIG. 10 (b) shows embodiments with N=2, equipped with a front insulator 35. FIG. 10 (c) shows an embodiment similar to (b) with the addition of a leading coil 36. The devices based on the principles of this invention can also be non-azimuthally symmetric and accordingly equipped with either an even or odd number of electrodes N.

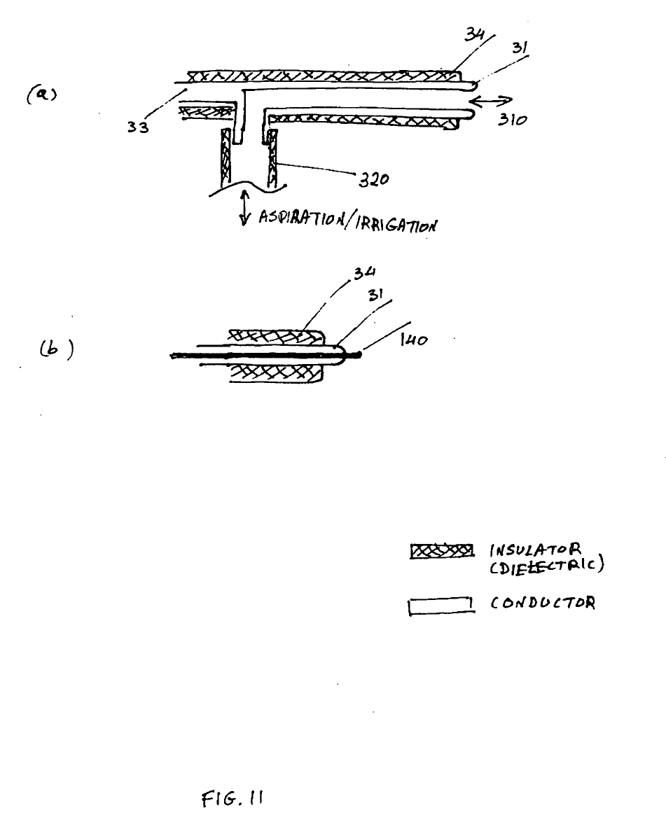

FIG. 11(a) schematically shows yet another embodiment of the device of the present invention, in which the active electrode 31 is equipped with an aspiration and or /irrigation port 310. It can be used, for example, to aspirate out debris from the active area or for introducing a cooling agent so as to reduce the local temperature in the active area. Both bipolar and monopolar devices aspiration/irrigation versions are contemplated herein. FIG. 11 (b) schematically shows an active electrode 31 based on the principles of this invention, equipped with a temperature-sensing element 140. Both monopolar and bipolar devices of this type are contemplated.

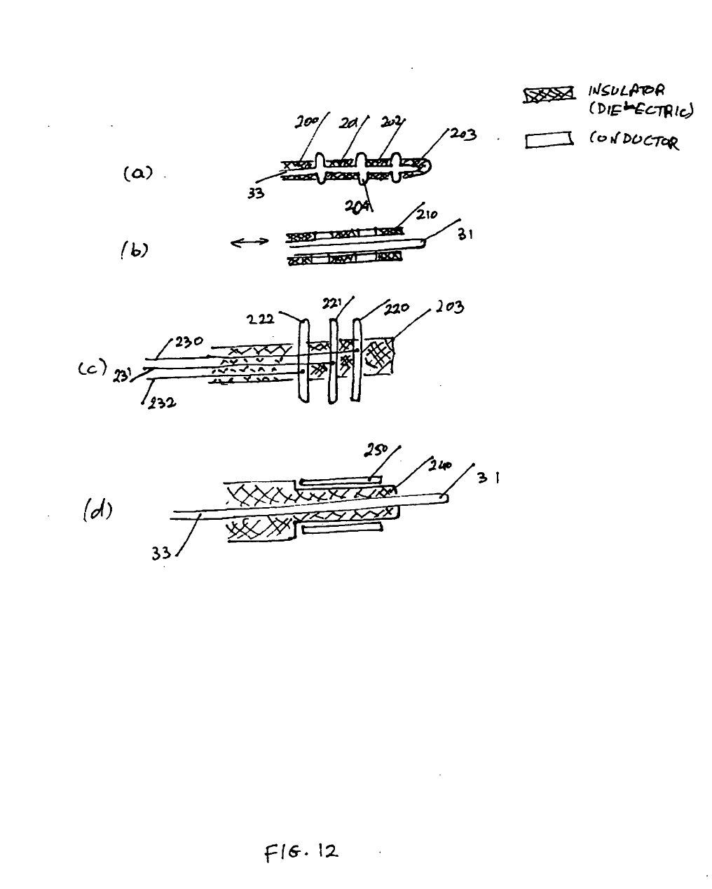

FIG. 12 shows still further embodiments of the device of the present invention. The active electrode 204 in FIG. 12 (a) is monopolar and includes multiple exposed protrusions or projections 200, 201, 202, and 203. The active electrode 204 is electrically connected to the external radio-frequency control unit (element 3 shown in FIG. 1) via an electrical connection 33. In FIG. 12 (b), the active electrode 31 is inserted into an insulator sleeve 210 which has one or more openings, symmetric on non-symmetric. The insulator 210 can be moved along and rotated around the electrode 31, thus exposing different segments depending on its relative position with respect to the electrode 31 depending on the specific needs of the patient. FIG. 12 (c) shows yet another monopolar embodiment equipped with three independent electrodes 220, 221 and 222, connected to an external radio-frequency unit with wires 230, 231 and 232. The independent electrodes can be activated simultaneously, sequentially or independently depending of the desired clinical effect. Both monopolar and bipolar devices of this type are possible based on the principles of this invention. FIG. 12 (d) is an example of a device equipped with one or more electrically detached electrodes 250 (i.e., electrodes not connected to an external source of electric energy). The detached electrode 250 is insulated from the active electrode 31 with an insulator 240. Note that the devices can also be of non-azimuthally symmetric designs, and equipped with a front guide coil. Other variations according to the principles of this invention are possible.

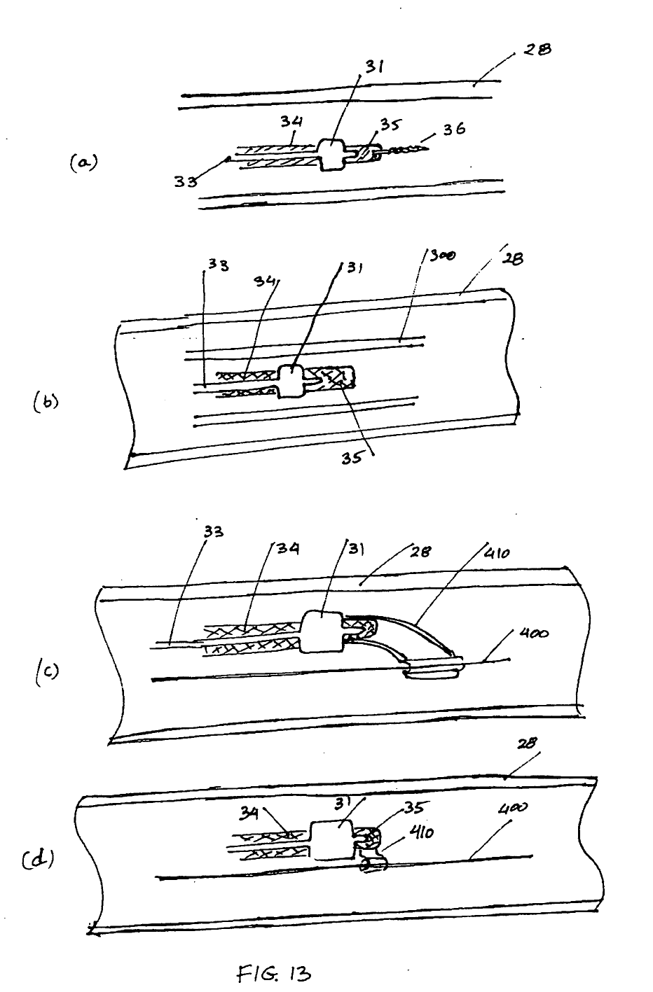

FIG. 13 illustrates exemplary methods for guiding the radio-frequency device of the present invention for passivation of vascular plaque inside blood vessels. In FIG. 13 (a), the device is equipped with a front insulator 35 and a guide coil 36. In FIG. 13 (b), the active electrode 31 is disposed inside a flexible, non-conducting tube 300 which is then inserted into the blood vessel 28 to be treated, and in this case a front guide coil is not needed. Both monopolar and bipolar devices of this type are contemplated, as are azimuthally symmetric and non-symmetric designs, with and without guide coils. In FIG. 13 (c), the active electrode 31 glides along a guide wire 400 with the aid of a sliding fixture 410, made of an insulating, flexible material, attached to the front end of active electrode 31. The device in FIG. 13 (d) is similar to the one shown in FIG. 13 (c), with the exception that the active electrode 31 glides along a guide wire 400 with the aid of a sliding fixture 420, made of an insulating material, attached to the side or front end of 31.

### REFERENCES

The disclosure of each publication, patent or patent application mentioned in this specification is specifically incorporated by reference herein in its entirety.
1. Murray C J L, Lopez A D. The global burden of disease. Global burden of disease and injury series. Boston, Mass.: Harvard School of Public Health/Harvard University Press, 1996:1-34.
2. Goldman L, Cook E F. The decline in ischemic heart disease mortality rates. An analysis of the comparative effects of medical interventions and changes in lifestyle. Ann Intern Med. 101:825-836, 1984.
3. Constantinides P. Plaque fissure in human coronary thrombosis. J Atheroscler Res. 6: 1-17, 1966.
4. Davies M J, Thomas A. Thrombosis and acute coronary-artery lesions in sudden cardiac ischemic death. New Engl J Med. 310:1137-1140, 1984.
5. Farb A. Tang A L, Burke A P Virmani R. Sudden coronary death: frequency of active coronary lesions, inactive coronary lesions, and myocardial infarction. Circulation 92: 1701-1709, 1995.
6. Davies M J, Bland J M, Hangartener J R W, Angelini A, Thomas A C. Factors influencing the presence or absence of acute coronary artery thrombi in sudden cardiac ischemic death. Eur Heart J. 10: 203-208, 1989.
7. Felton C V, Crook D, Davies M J, Oliver M F. Relation of plaque lipid composition and morphology to the stability of human aortic plaques. Arterioscler Thromb Vasc Biol. 17:1337-451997.
8. Moreno P R, Falk E, Palacios I F, Newell J B, Fuster V, Fallon J T. Macrophage infiltration in acute coronary syndromes. Implications for plaque rupture. Circulation 90: 775-81994.
9. Lendon C L, Davies M J, Born G V, Richardson P D. Atherosclerotic plaque caps are locally weakened when macrophages density is increased. Atherosclerosis 87:87-90, 1991.
10. Shah P K, Falk E, Badimon J J, et al. Human monocyte-derived macrophages induce collagen breakdown in fibrous caps of atherosclerotic plaques. Potential role of matrix-degrading metalloproteinases and implications for plaque rupture. Circulation 92: 1565-9, 1995.
11. Burke A P, Farb A, Malcom G T. Liang Y H, Smialek J, Virmani R. Coronary risk factors and plaque morphology in men with coronary disease who died suddenly. N Engl J Med 336:1276-82, 1997.
12. Farb A Burke A P, Tang A L, et al. Coronary plaque erosion without rupture into a lipid core. A frequent cause of coronary thrombosis in sudden coronary death. Circulation 93:1354-63, 1996.
13. Stary H C, Chandler A B, Dinsmore R E, Fuster V, Glagov S, Insull M E, Rosenfeld M E. Schwartz C J, Wagner W D, Wissler R W. A definition of advanced types of atherosclerotic lesions and a histological classification of atherosclerosis. A report from the Committee on Vascular Lesions of the Council on Arteriosclerosis. American Heart Association. Circulation 92:1355-74, 1995.
14. Virnani R, Kolodgie F D, Burke A P, Farb A, Schwartz S M. Lessons from sudden coronary death: a comprehensive morphological classification scheme for atherosclerotic lesions. Arterioscier Thromb Vasc Biol 20:1262-75, 2000.
15. Hunt J L, Fairman R, Mitchell M E, Carpenter J P, Golden M, Khalapyan T, Wolfe M, Neschis D, Milner R, Scoll B, Cusack A, Mohler E R. Bone formation in carotid plaques: a clinicopathological study. Stroke 33:1214-9, 2002.
16. Jeziorska M, McCollum C. Wooley D E. Observations on bone formation and remodelling in advanced atherosclerotic lesions of human carotid arteries. Virchows Arch 433:559-65, 1998.
17. Boddie A W Jr, Yamanashi W S, Frazer J, McBride C M, Martin R. Field focusing and focal heating patterns using a hybrid radiofrequency hyperthermia system. Med Instrum. 17:358-64, 1983.
18. Yamanashi W S, Boddie A W Jr, Frazer J W, McBride C M, Martin R G. Further observations on tissue heating patterns using an invasive ground probe with radiofrequency hyperthermia system. Med Instrum. 18:220-3, 1984.
19. Huang S K, Graham A R, Hoyt R H, Odell R C. Transcatheter desiccation of the canine left ventricle using radiofrequency energy: a pilot study. Am Heart J. 114:42-8, 1987.
20. Huang S K, Bharati S. Graham A R, Lev M, Marcus F I, Odell R C. Closed chest catheter desiccation of the atrioventricular junction using radiofrequency energy—a new method of catheter ablation. J Am Coll Cardiol. 9:349-58, 1987.
21. LePage J R, Lewis J E, Ruiz O F, Yamanashi W S, Padron G M, Hood C H. Angiopyroplasty using electromagnetically induced focused heat. Angiology. 38:520-3, 1987.
22. Sigel B, Dunn M R. The mechanism of blood vessel closure by high frequency electrocoagulation. Surg Gynecol Obstet. 121:823-31, 1965.

While the invention has been described with reference to specific examples and preferred embodiments, it will be appreciated that the description is illustrative of the invention and, therefore, should not be constructed as limiting thereof. It should also be understood that the invention is intended not to be limited by the foregoing description, but to be defined by the appended claims and their equivalents. Various modifications and applications may occur to those who are skilled in the art, without departing from the spirit and the scope of the invention, as described by the appended claims.

What is claimed:

1. A method of local or regional vascular therapy for atherosclerotic, inflammatory, and/or vulnerable plaque in a blood vessel in a subject in need thereof, said method comprising the steps of:
(a) introducing a radio-frequency device having one or more active electrodes at its distal end into a blood vessel;
(b) positioning one of said active electrodes in close proximity to said plaque;
(c) applying low power radio-frequency energy to the active electrode for a controlled amount of time so as to passivate said plaque by selectively heating the plaque while minimizing the heat generated in the blood vessel wall and surrounding tissue, wherein said selective heating results in transformation of the plaque into mechanically stable fibrotic lesions having a reduced risk of thrombosis.

2. The method of claim 1 further comprising the step of (d) repositioning the device during step (c) so as to enhance passivation.

3. The method of claim 2, wherein said repositioning step (d) involves manual manipulation.

4. The method of claim 2, wherein said repositioning step (d) is automated.

5. The method of claim 2, wherein said repositioning step (d) comprises translational or rotational movement or a combination of the two.

6. The method of claim 2, wherein said repositioning step (d) comprises continuous or interrupted movement.

7. The method of claim 2, wherein said repositioning step (d) involves oscillating the device back and forth over a central plaque position.

8. The method of claim 1, wherein said plaque is disposed in a leg vessel.

9. The method of claim 1, wherein said plaque is disposed in a cardiac vessel.

10. The method of claim 1, wherein said plaque is non-symmetrically disposed within the blood vessel.

11. The method of claim 1, wherein said active electrode is provided with a sensor at its distal end.

12. The method of claim 11, wherein said sensor is a temperature sensor and said method further comprises the step of monitoring the temperature at the active site and adjusting the power level as needed to achieve selective heating and avoid injury to the blood vessel and surrounding tissue.

13. The method of claim 12, wherein said sensor is an energy or impedance sensor.

14. The method of claim 1, wherein said device comprises an aspiration lumen terminating in an aspiration port disposed at the distal end of said device, further wherein said method further comprises the step of aspirating debris from the treatment site through said aspiration port and aspiration lumen.

15. The method of claim 1, wherein said device comprises an irrigation lumen terminating in an irrigation port disposed at the distal end of said device, further wherein said method further comprises the step of introducing cooling fluid via said irrigation lumen and irrigation port to the treatment site.

16. The method of claim 1, wherein said device comprises an irrigation lumen terminating in an irrigation port disposed at the distal end of said device and an aspiration lumen terminating in an aspiration port disposed at the distal end of said device, further wherein said method comprises the steps of (i) introducing cooling fluid to the treatment site via said irrigation lumen and irrigation port and (ii) aspirating debris from the treatment site through said aspiration port and aspiration lumen.

17. The method of claim 1, wherein said radio-frequency device comprises at least one active electrode disposed within an elongated insulator sleeve, said sleeve provided with one or more lateral openings at its distal end.

18. The method of claim 17, wherein said active electrode extends through said one or more lateral openings.

19. The method of claim 1, wherein said radio-frequency device comprises at least one active electrode that protrudes beyond the distal end of an elongated electrical insulator sleeve, wherein at least one conductive wire, connected to one of said active electrodes at one end and an external radio-frequency control unit at its other end, is disposed within said insulator sleeve.

20. The method of claim 19, wherein said insulator sleeve is made from a flexible dielectric material.

21. The method of claim 20, wherein said material is plastic.

22. The method of claim 19, wherein the distal end of said insulator sleeve is slidably disposed about said active electrode and is provided with a series of lateral openings that allow for control of the direction of radio-frequency energy applied to said plaque.

23. The method of claim 19, wherein said radio-frequency device comprises two or more pairs of active bipolar electrodes disposed within said insulator sleeve.

24. The method of claim 19, wherein said device further comprises a second bipolar electrode coaxially disposed about said insulator sleeve.

25. The method of claim 24, wherein said second electrode is provided with a separate insulative dielectric coating.

26. The method of claim 24, wherein said second electrode is mounted to the distal end of said insulator sleeve and disconnected from the external source of radio-frequency energy.

27. The method of claim 19, wherein the diameter of said active electrode is greater than the diameter of said conductive wire.

28. The method of claim 19, wherein the diameter of said active electrode is greater than the diameter of said insulator sleeve.

29. The method of claim 19, wherein said active electrode is slidably disposed within said insulator sleeve.

30. The method of claim 1, wherein said active electrode is provided with an insulated distal tip.

31. The method of claim 30, wherein said insulated tip is provided with a front guide coil that facilitates navigation of the device inside the subject's blood vessels.

32. The method of claim 2, wherein introduction step (a) and positioning steps (b) and (d) are monitored using an external imaging system.

33. The method of claim 32, wherein said imaging system comprises a fluoroscope.

34. The method of claim 1, wherein the power applied is less than 50 W.

35. The method of claim 34, wherein the power applied is less than 25 W.

36. The method of claim 35, wherein the power applied is less than 10 W.

37. The method of claim 1, wherein the radio-frequency energy applied ranges from 100 kHz to 10 MHz.

38. The method of claim 37, wherein the radio-frequency energy applied ranges from 300 kHz to 6 MHz.

39. The method of claim 1, wherein said radio-frequency energy is applied for 20 seconds or less per treatment site.

40. The method of claim 1, wherein said active electrode is a monopolar electrode.

41. The method of claim 1, wherein said active electrode is a bipolar electrode.

42. The method of claim 1, wherein said radio-frequency device is introduced into the blood vessel through a central lumen of a flexible guide tube.

43. The method of claim 1, wherein said radio-frequency device is introduced into the blood vessel over a flexible guide wire.

44. The method of claim 1, wherein the distal end of said active electrode is provided with a sliding fixture that slides over said guide wire.

45. A radio-frequency device for passivation of vascular plaque comprising: a first active electrode connected to a conductive wire, an insulator sleeve coaxially disposed about said conductive wire, and a second active electrode coaxially disposed and mounted to the distal end of said insulator sleeve yet disconnected from the external source of radio-frequency energy, wherein the distal end of said first electrode extends beyond the distal ends of said insulator sleeve and second electrode.

46. A radio-frequency device for passivation of vascular plaque comprising: a first active electrode connected to a conductive wire and an insulator sleeve disposed about said electrode, wherein the distal end of said insulator sleeve is slidably disposed about said active electrode and is provided with a series of lateral openings that control of the direction of energy applied from the active electrode to said plaque.

47. A radio-frequency device for passivation of vascular plaque comprising: an active electrode connected to a conductive wire and an insulator sleeve disposed about said conductive wire, wherein said insulator sleeve is provided with one or more lateral openings and said active electrode includes one or more projections, each of which extend through one of said lateral openings.

48. The radio-frequency device of claim 47, wherein said lateral openings are circumferentially disposed about said electrode.

49. The radio-frequency device of claim 47, wherein said lateral openings comprise side ports that do not extend around the circumference of the electrode.

50. The radio-frequency device of claim 47, wherein said electrode projections extend beyond the outer diameter of said insulator sleeve.
